(19) United States
(12) Patent Application Publication
(10) Pub. No.: US 2026/0053464 A1
(43) Pub. Date: Feb. 26, 2026

(54) AUTOMATED ECHOCARDIOGRAM PROCESSING TO CHARACTERIZE SHUNTS

(71) Applicant: Agitated Solutions Inc., St. Paul, MN (US)

(72) Inventors: Morgan Evans, Apple Valley, MN (US); Micah Eimer, Glenview, MN (US); Jennifer Chmura, Minneapolis, MN (US); Ryan Kruchten, Denver, CO (US)

(73) Assignee: Agitated Solutions Inc., St. Paul, MN (US)

(21) Appl. No.: 19/378,271

(22) Filed: Nov. 3, 2025

Related U.S. Application Data
(63) Continuation of application No. 18/131,349, filed on Apr. 5, 2023, now abandoned.
Application No. 18/131,349 is a continuation-in-part of application No. 17/321,957, filed on May 17, 2021, now Pat. No. 11,986,342.
(60) Provisional application No. 63/026,177, filed on May 18, 2020.
(60) Provisional application No. 63/327,738, filed on Apr. 5, 2022.

(51) Int. Cl. A61B 8/06; A61B 8/00; A61B 8/12; A61B 17/00; A61K 49/22

(52) U.S. Cl. CPC A61B 8/06; A61B 8/481; A61B 8/5223; A61B 17/0057; A61K 49/223; A61B 8/12; A61B 2017/00575

(57) ABSTRACT

A method may include performing an echocardiogram procedure on a patient to generate a plurality of echocardiogram images; during the echocardiogram procedure, intravenously directing bubbles into a patient's venous circulatory system and to a right side of the patient's heart; identifying structures of the patient's heart and further identifying composite boundaries for the structures; after detecting presence of the bubbles on the right side of the patient's heart, comparing opacification of the right atrium to a threshold level of opacification to confirm sufficient opacification of the patient's right atrium; analyzing additional echocardiogram images relative to the composite boundaries to detect presence of the bubbles on a left side of the patient's heart; based on analyzing the additional echocardiogram images, calculating a score based on a nature of detected bubbles on the left side of the patient's heart; and based on the score initiating treatment in the patient.

### CROSS-REFERENCE TO RELATED APPLICATIONS

This application is a continuation of U.S. application Ser. No. 18/131,349, titled “Automated Echocardiogram Processing to Characterize Shunts,”filed Apr. 5, 2023. U.S. application Ser. No. 18/131,349 is a continuation-in-part of U.S. application Ser. No. 17/321,957, titled “Characterization of Cardiac Shunts with Bubbles,” filed May 17, 2021, now U.S. Pat. No. 11,986,342, and further claims the benefit of U.S. Provisional Application Ser. No. 63/327,738, titled “Image Processing for Assessing Shunts”, filed Apr. 5, 2022. U.S. application Ser. No. 17/321,957 claims the benefit of U.S. Provisional Application Ser. No. 63/026,177, titled “Characterization of Cardiac Shunts with Bubbles,” filed May 18, 2020. This application incorporates the entire contents of the foregoing applications herein by reference.

### TECHNICAL FIELD

Various embodiments relate generally to diagnosing and characterizing cardiac shunts.

### BACKGROUND

A patent foramen ovale (PFO) or atrial septal defect (ASD) may be present in the septum that would otherwise separate two sides of a patient's heart. When present, either a PFO or an ASD can allow embolic material—which would otherwise be filtered out by the patient's lungs but for the PFO or ASD—to enter arterial circulation.

Paradoxical embolization through a patent foramen ovale (PFO) may be implicated in cryptogenic strokes when there is a prevalent right-to-left shunt. The causal relationship between PFOs and a stroke is controversial; although the prevalence of PFOs in the adult population is high (25% of adults in the US), presence of a PFO is not indicative of an increased risk of stroke. However, patients with a prior cryptogenic stroke have a significantly higher presence of PFOs (68%).

Patients may benefit from interventions including endovascular or surgical closure, anticoagulation, or antiplatelet therapy. Device PFO closure has found to have recurrent stroke in 0-5% of patients, major procedural complications in 1.5% of patients, and minor complications in 7.9% of patients.

Evaluating the risk-benefit of PFO treatment is difficult and wrought with anxiety for patient and provider. Between the commonality of PFOs, the procedural risk of closure, and the severity and high incidence of strokes, there is currently no scientific consensus on identifying patients in which PFO closure would be beneficial from those where it would be detrimental. Guidance on when to pursue these interventions is lacking due to diagnostic limitations for grading whether the PFO had or will have a causal role in stroke.

Transesophageal or transthoracic echocardiography (TEE or TTE) imaging with agitated saline bubbles as a contrast is the gold standard test for evaluating the presence of PFOs following stroke. Bubble studies use cardiac cycle timing of bubbles appearing from the right (RA) to left atrium (LA) to distinguish intracardiac abnormalities such as PFO from transpulmonary shunts (TPS). Three-dimensional TEE can provide some morphological features of the PFO, including tunnel length, margins or rims, and anatomical detail of surrounding structures such as the aorta and inferior vena cava. Despite the wealth of information provided by TEE, it is not readily implemented in diagnosis of PFO-associated strokes given (1) acquisition difficulties and (2) procedural constraints. Acquisition difficulties arise due to how contrast enhancement is delivered in bubble studies in standard of care (SOC), which uses the Tessari's method to agitate air and fluid (typically saline or glucose) and manual injections.

### SUMMARY

A method may include performing an echocardiogram procedure on a patient to generate a plurality of echocardiogram images; during the echocardiogram procedure, intravenously directing bubbles into a patient's venous circulatory system and to a right side of the patient's heart; prior to detecting presence of the bubbles on the right side of the patient's heart, and from a subset of echocardiogram images within the plurality of echocardiogram images, each echocardiogram image in the subset corresponding substantially to a same point in different cardiac cycles, identifying structures of the patient's heart and further identifying composite boundaries for the structures; after detecting presence of the bubbles on the right side of the patient's heart, comparing opacification of the right atrium to a threshold level of opacification to confirm sufficient opacification of the patient's right atrium; analyzing additional echocardiogram images in the plurality of echocardiogram images relative to the composite boundaries to detect presence of the bubbles on a left side of the patient's heart; based on analyzing the additional echocardiogram images, calculating a score based on a nature of detected bubbles on the left side of the patient's heart; and based on the score initiating treatment in the patient.

The generated plurality of echocardiogram images may be associated with different points within a cardiac cycle of the patient, across multiple cardiac cycles of the patient. The identified structures may include at least a left atrium, a right atrium and an atrial septum. A higher value for the score may correspond to a relatively more severe shunt, and a lower value for the score may correspond to a relatively less severe shunt. Initiating treatment may include, (i) for a high score, surgically closing the shunt; (ii) for a low score, treating the patient with blood thinners; and (iii) for an intermediate score, performing a follow-on transesophageal echocardiogram (TEE) study.

Intravenously directing the bubbles may include injecting an agitated solution of saline or dextrose into a median cubital vein of the patient. Performing an echocardiogram procedure may include performing either a transthoracic echocardiogram (TTE) or a TEE. Surgically closing the shunt in the patient's heart may include using a catheter-delivered percutaneous closure device. The method may further include determining a blood flow rate, using ultrasound imaging, by monitoring movement of the bubbles within the patient's heart.

Analyzing the additional echocardiogram images relative to the composite boundaries may include segmenting a region of interest and detecting the bubbles in one or more segments within the region of interest. The region of interest may include a portion of the left atrium, and a component of the calculated score may be associated with proximity of detected bubbles relative to the atrial septum; the component may be higher for detected bubbles farther from the atrial septum than for detected bubbles that are closer to the atrial septum. The region of interest may include a portion of the left atrium, and a component of the calculated score may be based on a time analysis of detected bubbles; the component may be higher for a bolus of detected bubbles within a smaller window of time relative to a smaller number of detected bubbles over a longer window of time. The additional echocardiogram images may include echocardiogram images that are captured within three to five cardiac cycles of when the bubbles are detected on the right side of the patient's heart.

Identifying the structures of the heart may further include identifying a left ventricle; the region of interest may include the left ventricle; a component of the score may be based on whether bubbles are detected in the left ventricle; and the component may be higher when the bubbles are detected in the left ventricle and lower when the bubbles are not detected in the left ventricle.

Confirming sufficient opacification of the patient's right atrium may further include confirming sufficient opacification adjacent the atrial septum. Confirming sufficient opacification of the patient's right atrium may further include confirming absence of negative contrast adjacent the atrial septum.

The method may further include confirming proper execution of a Valsalva maneuver.

Confirming proper execution of a Valsalva maneuver may include confirming, using at least two images in the echocardiogram images, that the atrial septum has shifted from the right atrium toward the left atrium. Confirming proper execution of a Valsalva maneuver may include confirming, using at least two images in the echocardiogram images, that the right atrium has increased in volume or that the left atrium has decreased in volume.

### BRIEF DESCRIPTION OF THE DRAWINGS

FIG. 1 illustrates a portion of a human circulatory system.

FIG. 2A illustrates detail of a human heart.

FIG. 2B depicts a ventricular systole phase of a heart.

FIG. 2C depicts an atrial systole phase of a heart.

FIGS. 3A-3C depict bubble studies conducted with bubbles of varying sizes.

FIG. 4 illustrates a method for conducting multiple bubble studies in sequence to characterize a cardiac shunt.

FIG. 5A illustrates an exemplary ECG waveform for a patient undergoing a bubble study.

FIGS. 5B-1 and 5B-2 illustrate echocardiogram images corresponding to the ECG of FIG. 5A.

FIG. 5C depicts a process for identifying regions of interest in echocardiogram images.

FIGS. 5D-1 and 5D-2 depict a process for determining average boundaries for exemplary regions of interest.

FIGS. 5E-1 and 5E-2 depict a method of applying average boundaries to echocardiogram images, to facilitate further analysis.

FIG. 5F depicts an exemplary method for segmenting a right atrium image and analyzing to confirm sufficient opacification during a bubble study.

FIGS. 5G-1 and 5G-2 depict methods for analyzing the left side of a patient's heart during a bubble study to detect migration of bubbles through a shunt and to characterize the shunt.

FIG. 6A-1 illustrates an exemplary ECG waveform for a patient undergoing a bubble study.

FIG. 6A-2 illustrates echocardiogram images corresponding to the ECG of FIG. 6A-1.

FIG. 6A-3 depicts a process for identifying a region of interest in echocardiogram images shown in FIG. 6A-2.

FIG. 6B-1 illustrates an exemplary ECG waveform of a patient undergoing a bubble study, while performing a Valsalva maneuver.

FIG. 6B-2 illustrates echocardiogram images corresponding to the ECG of FIG. 6B-1.

FIG. 6B-3 depicts a process for identifying a region of interest in echocardiogram images shown in FIG. 6B-2.

FIG. 6C depicts comparison of average regions of interest between a bubble study without a Valsalva maneuver being performed, and a bubble study with a Valsalva maneuver being performed.

### DETAILED DESCRIPTION

Agitated saline contrast studies are a useful adjunct to many ultrasound examinations, particularly cardiac ultrasound (echocardiography). Injection of agitated saline into a vein combined with echocardiography may be used to detect shunts which may be within the heart, such as a patent foramen ovale (PFO) or an atrial septal defect (ASD) (two types of holes in the heart), or external to the heart (e.g., in the lungs) known as pulmonary arteriovenous malformations (pAVM). Agitated saline can also be used with echocardiography to confirm catheter placement in fluid around the heart (pericardiocentesis), detect anomalous connections within the heart, visualize the right side of the heart, and accentuate right-sided blood flow for the purpose of quantitation.

Agitated saline contrast echocardiography takes advantage of the increased reflection that results when ultrasound waves meet a liquid/gas interface. This allows for visualization of otherwise poorly reflective areas such as fluid filled cavities by ultrasound imaging equipment. Applications in which this has been clinically useful include echocardiography where agitated saline can be used to define the structural integrity of the interatrial septum or infer the presence of a transpulmonary shunt. Agitated saline can also be combined with Doppler echocardiography to assess blood flow through the tricuspid valve. An alternative method to detect atrial defects uses ultrasound of the brain vessels (transcranial Doppler) to detect bubbles that have crossed from the right heart to the left heart and entered the cerebral circulation.

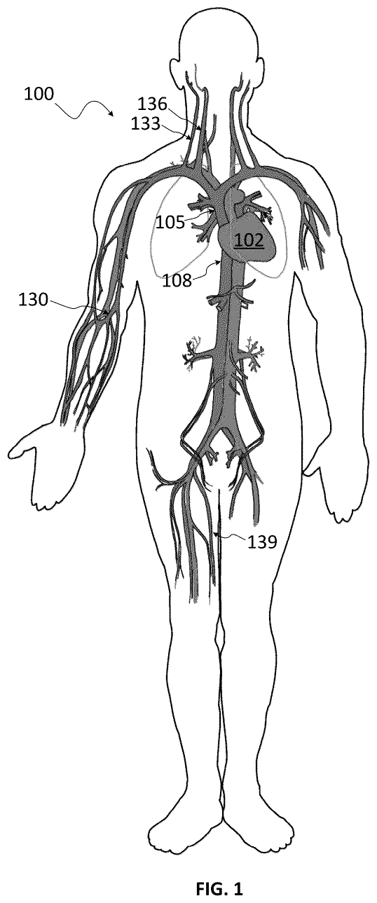

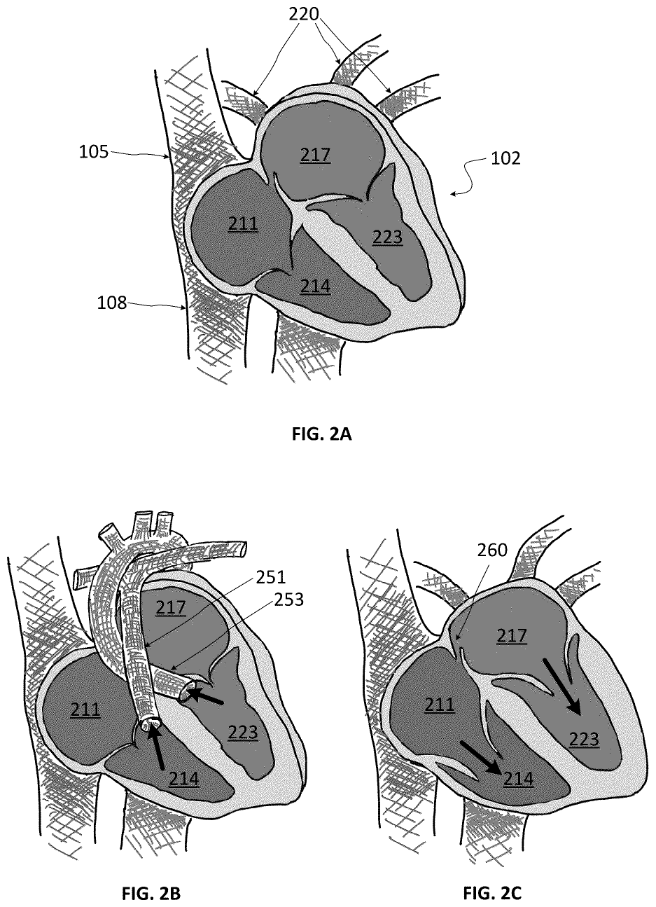

Described herein are methods for characterizing the extent of shunts, such as interatrial shunts. For context, various aspects of a human cardiovascular system are first described with reference to FIG. 1 and FIGS. 2A, 2B and 2C. FIG. 1 illustrates a portion of an overall human circulatory system 100. At its core, is the heart 102, and a system of arteries that extend from the heart, and veins that return to the heart.

Various internal structures of the heart 102 are described in greater detail with reference to FIG. 2A. Blood is returned to the heart 102 from throughout the body by the vena cava, which is divided into the superior vena cava 105, which collects blood from the upper portion of the body, and the inferior vena cava 108, which collects blood from the lower portion of the body. Blood flows through the superior vena cava 105 and inferior cava 108 on its way to the right atrium 211.

After being oxygenated in the lungs, blood is returned to the left atrium 217 of the heart 102 via the pulmonary veins 220 (three of four of which are shown). From the left atrium 217, the heart 102 pumps blood into the left ventricle 223, which in turns pumps it to the aorta for distribution throughout the body.

In more detail, with reference to FIG. 2B, the right ventricle 214 and left ventricle 223 contract during a ventricular systole phase, pumping blood from the right ventricle 214 into the pulmonary artery 251 and from the left ventricle 223 to the aorta 253. With reference to FIG. 2C, the left atrium 211 and right atrium 217 contract during an atrial systole phase, pumping blood from the right atrium 211 to the right ventricle 214 and from the left atrium 217 to the left ventricle 223. In FIG. 2C, the pulmonary artery 251 and aorta 253 have been removed to better illustrate intracardiac flow.

Also illustrated in FIG. 2C is a shunt 260 that fluidly couples the right atrium 211 and the left atrium 217. When present, such a shunt can increase a risk of stroke by allowing embolic material (e.g., a blood clot, a fat globule, other foreign material) to move from the right atrium 211 to the left atrium 217, then into the left ventricle 223, and finally into arterial circulation through the body via the aorta 253—where the embolic material can cause a stroke or other critical blockage. But for the presence of such a shunt 260, the embolic material would, in most patients, be filtered by the lungs.

Shunts may take different forms. For example, a PFO is a small flap-like opening that is normally present at birth in the heart wall (septum) that separates the left atrium from the right atrium. In some patients, this PFO never fully closes after birth. An ASD is a type of birth defect in which a hole exists in the septum dividing the atria. Often, an ASD is more serious than a PFO. Not illustrated, but also possible, is a ventricular septal defect (VSD), in which a hole exists in the septum that separates the right ventricle from the left ventricle.

When a shunt 260 is present in a patient's heart, the size of the shunt 260 may determine the risk of a traumatic or catastrophic effect of embolic material entering arterial circulation via the left atrium 217, left ventricle 223 and aorta 253, rather than being filtered out in the lungs. A larger shunt may, depending on other aspects of the patient's anatomy and physiology, allow larger emboli to enter arterial circulation, where such emboli could cause a stroke, heart attack or other traumatic blockage.

Many shunts can be treated—either surgically (e.g., with percutaneous closure using a catheter, or with open-heart surgery and direct surgical closure), or with medication (e.g., blood thinners, to reduce the risk of clots). However, many shunts need not be treated. That is, many shunts are small enough, and patients may be otherwise healthy enough that the reduction of risk by closing a small shunt may be outweighed by the risk of a procedure to close the shunt.

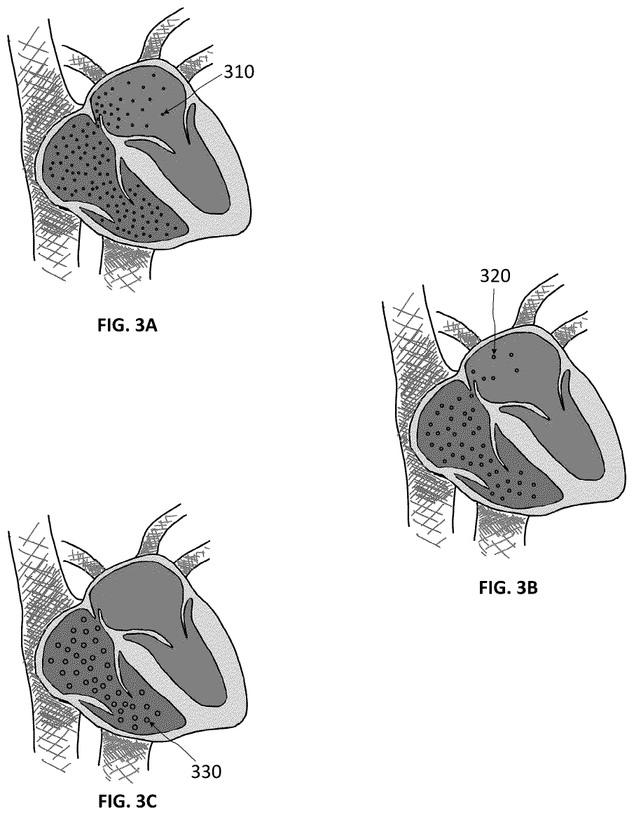

For example, in some patients with ASDs, where the ASD is less than 5 mm and there is no evidence of either right ventricular volume overload or paradoxical embolism, it may be safer to not surgically treat the ASD; on the other hand, ASDs that are larger than 5 mm or in patients where right ventricle overload is detected, or where the patient has suffered a cryptogenic stroke, surgical repair of the ASD may be indicated. Similarly, in some patients, surgical repair may be indicated for PFOs that exceed 4 mm in diameter, or in patients that have recently suffered a cryptogenic stroke. For these reasons, it can be advantageous to accurately assess the size of the shunt, to facilitate weighing of risks between repairing an ASD or PFO or leaving it untreated surgically.

Whereas a single bubble study may be used to assess the presence of a cardiac shunt, additional diagnostic information can be obtained from multiple bubble studies, especially when it is possible to control for bubble size and progressively and predictably increase the size of bubbles from one bubble study to the next. A method for assessing size of a cardiac shunt is now described with reference to FIG. 3A, FIG. 3B and FIG. 3C.

As depicted in FIG. 3A, a bubble study may be conducted using small bubbles 310 (e.g., bubbles having a diameter in the approximate range of 8 um to 15 um, where “approximate” may mean within 1%, 5%, 10%, 20%, 25% or 50% of a nominal value). Upon confirmation of a shunt (e.g., by detection of immediate (e.g., within one, two or three atrial systole phases) bubble migration from right atrium to left atrium, a second bubble study may be conducted using larger bubbles 320 (e.g., bubbles having a diameter in the approximate range of 15 um to 25 um), as depicted in FIG. 3B. In some cases, regardless of the outcome the second bubble study, a third bubble study, using still larger bubbles 330 (e.g., bubbles having a diameter in the approximate range of 25 um to 35 um), as depicted in FIG. 3C, may be conducted; in other cases, the third bubble study may only be conducted if a shunt is confirmed by the second bubble study.

Described above is a sequence of bubble studies in which the first study is with “small” bubbles and the last study is conducted with “large”bubbles. This order may also be reversed. For example, in some patients where a cardiac shunt is anticipated (e.g., following a cryptogenic stroke), a “large” bubble study may be conducted first. In such a case, if a suspected cardiac shunt is confirmed, no additional bubble studies may be required. Three bubble studies are described above, but in some cases, even when the first study involves small bubbles, only two bubble studies may be required. In some cases, multiple (e.g., two, three, four or more) bubble studies may be conducted using the same size bubbles. In some cases, different size bubbles may be used; for example, when the risk of inducing an air embolism is determined to be low for a specific patient, bubbles larger than 35 um may be employed. The methods described herein may be modified in order, repetition and in other ways, at the discretion of the medical care provider.

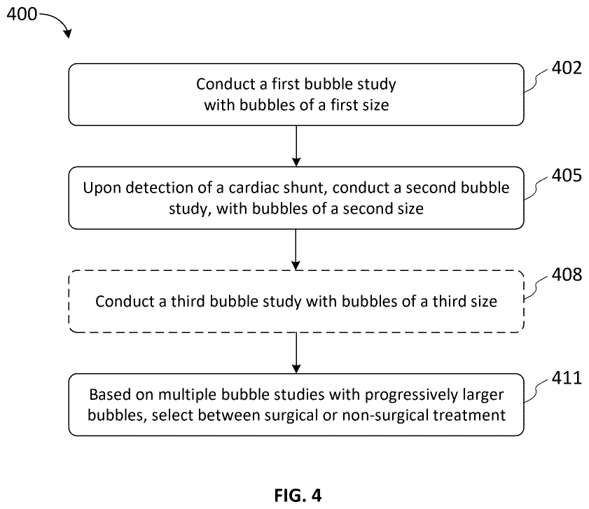

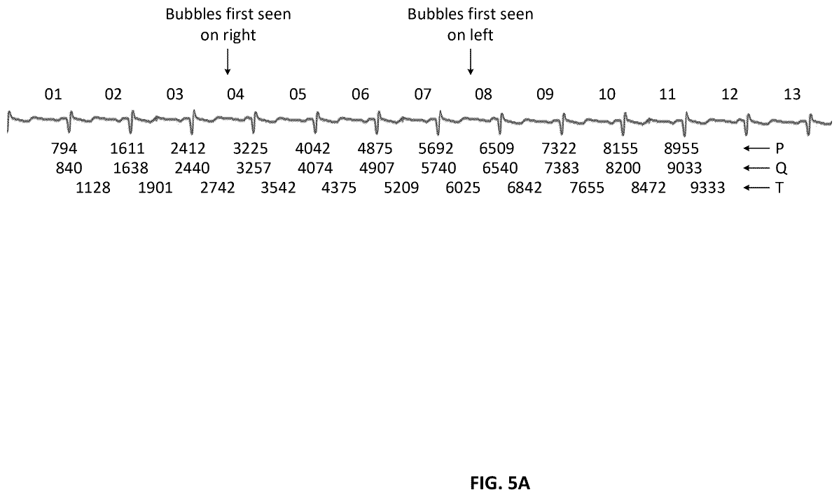

FIG. 4 illustrates a method 400 for conducting a sequence of bubble studies. The method 400 includes conducting (402) a first bubble study with bubbles of a first size. For example, a bubble study with small bubbles 310, such as depicted in FIG. 3A, may be first conducted (402) to identify a shunt of any significant size. The first bubble study may be motivated by a stroke (e.g., a cryptogenic stroke, or stroke of unknown causes), or it may be part of a cardiac workup that is motivated by other reasons.

When a shunt is detected, for example by detection of bubbles on the left side of the heart (either in the left atrium or left ventricle) within one systole phase of their presence on the right side of the heart (or, in some implementations, within two or three atrial systole phases), the method 400 includes conducting (405) a second bubble study with bubbles of a second size. For example, a bubble study with larger bubbles 320, as depicted in FIG. 3B, may be conducted (405). Detection of these larger bubbles 320 on the left side of the heart can provide further information about the nature and extent of the shunt.

Optionally (and as a matter of course, in some cases, when bubbles in the second bubble study are detected on the left side of the heart), a third bubble study may be conducted (408) with still-larger bubbles. For example, the third bubble study may employ still-larger bubbles 330.

Based on the multiple studies with progressively larger bubbles, it may be possible to characterize a size or extent of a cardiac shunt; based on this characterization, the method 400 can include selecting (411) between surgical or non-surgical treatment of the shunt.

For example, if a first bubble study with small bubbles, such as the study depicted in FIG. 3A, identifies a shunt in a patient's heart, but bubbles are not detected on the left side of the patient's heart in a follow-on study with larger bubbles, and the patient has no other underlying health concerns that would make an unclosed shunt particularly high-risk, the shunt may be left untreated surgically. In some cases, the patient may be provided with a blood thinner, to reduce the risk of clots, or the patient may be provided with some other prophylactic medication. As another example, if bubble migration from right to left were detected in a patient in three bubble studies with progressively larger bubbles, it may be determined that a shunt of significant size is present, and surgical closure may be indicated.

By controlling the size of the bubbles (e.g., by generating them in a manner that results in relatively consistent and fixed sizing—for example, one in which a median bubble size may fall within a specified range, or in which a majority of bubbles within a distribution of bubble sizes may fall within one, two or three standard deviations of a specified range) and by measuring or inferring other cardiac parameters, such as blood volume and blood flow, it may be possible to characterize cardiac shunts in far greater detail than would otherwise be possible. In addition to detecting mere presence of bubbles of any given size, a volume or number of bubbles may also be measured. In this manner, particularly when bubble volume, blood volume and blood flow are all measured or inferred, it may be possible to characterize larger shunts, even if it is not possible (e.g., for safety reasons) to use very large bubbles alone to character such shunts.

Multiple techniques may be employed to detect the presence of bubbles on the left side of the heart. For example, a noninvasive transthoracic echocardiogram (TTE) may be employed, whereby an ultrasound transducer is placed on the chest of a patient undergoing the bubble study. High frequency soundwaves (ultrasound) are used to create a moving picture of the heart, through the chest wall, and when the ultrasound and bubble study are properly performed, bubbles that are present on either side of the heart will be picked up and imaged through the procedure.

A transesophageal echocardiogram (TEE) may also be employed for higher resolution of images. In a TEE, and ultrasound transducer is placed in the esophagus of the patient undergoing the procedure. Given the proximity of the esophagus to the heart, and given that the ultrasound in a TEE does not have to traverse the chest wall and rib cage, the images from a TEE are typically much clearer than with a TTE.

Machine learning algorithms may be employed across multiple bubble studies to provide additional diagnostic information. For example, precise measurements could be captured from the ultrasound images in ether TTE or TEE procedures, to determine blood volume in each chamber of a patient's heart. Individual bubbles could be traced to capture a blood flow rate.

Regression analysis could be applied across many patients to determine likelihood of bubbles (or bubbles of a particular size) appearing on the left side of the patient's heart when a shunt is present, or a shunt of a particular kind, given specific volumes or flow rates. When such a shunt is identified and repaired, more precise information about its size could be gleaned during the procedure for its repair, and this information could be fed back into the machine learning algorithm. Other variables could be incorporated into such machine learning algorithms (e.g., patient age, other heart or general health conditions, respiratory function, gender, genetics, blood type, etc.).

To facilitate bubble studies or other diagnostic or therapeutic procedures whereby bubbles are to be introduced into the circulatory system, one must get the bubbles into the venous system and ultimately into the superior vena cava 105 or inferior vena cava 108, and into the right atrium 211 of the heart 102. With reference to FIG. 1, there are several common access points through which bubbles can be so introduced. Common among them is intravenous introduction of bubbles (e.g., mixed with saline or dextrose) via the median cubital vein 130 of the right arm. From here, blood flows through the basilic vein, axillary vein, subclavian vein, and into the superior vena cava 105. Alternative paths to the superior vena cava 105 are the external jugular vein 133, or internal jugular vein 136, both of which drain into the brachiocephalic vein prior to reaching the superior vena cava 105. An alternative inferior route includes the femoral vein 139, which flows into the inferior vena cava 108 prior to reaching the right atrium 211. Other routes to the right atrium 211 are possible.

### Image Processing Using Artificial Intelligence/Machine Learning

Instead of cardiologists counting microbubbles, the culpability of thrombus embolization through a PFO could instead theoretically be quantified by the time-course of contrast enhancement in the LA/RA with artificial intelligence (AI).

Application of AI to echocardiography broke ground in early 2020, when the FDA authorized Caption Guidance software which collects cardiac US data for tasks including classification of standard US videos, automated segmentation, left ventricular volume calculations, and diagnosis. The intention behind utilizing AI in US is to automate otherwise complex processes that require significant time and thus cost from echocardiographers, as well as reduce user subjectivity for more accurate and standardized analysis. Deep learning models are a growing subfield which use layers of algorithms within an artificial neural network to enable the AI software to learn input/output translation without supervision. Using TEE US microbubble studies as an input into a machine learning algorithm that outputs PFO or intra-pulmonary shunt (IPS) detection, as well as an ordinal score of PFO risk for recurrent stroke, would revolutionize evidence-based practice in PFO assessment. Outlined herein is a process for applying machine learning to improve the diagnostic value of bubble studies.

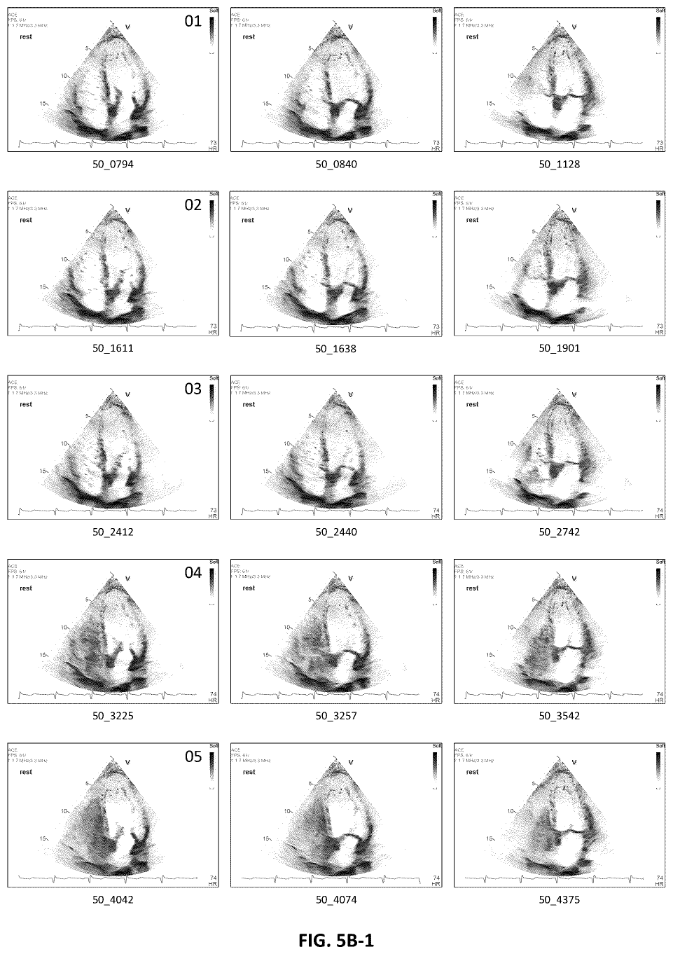

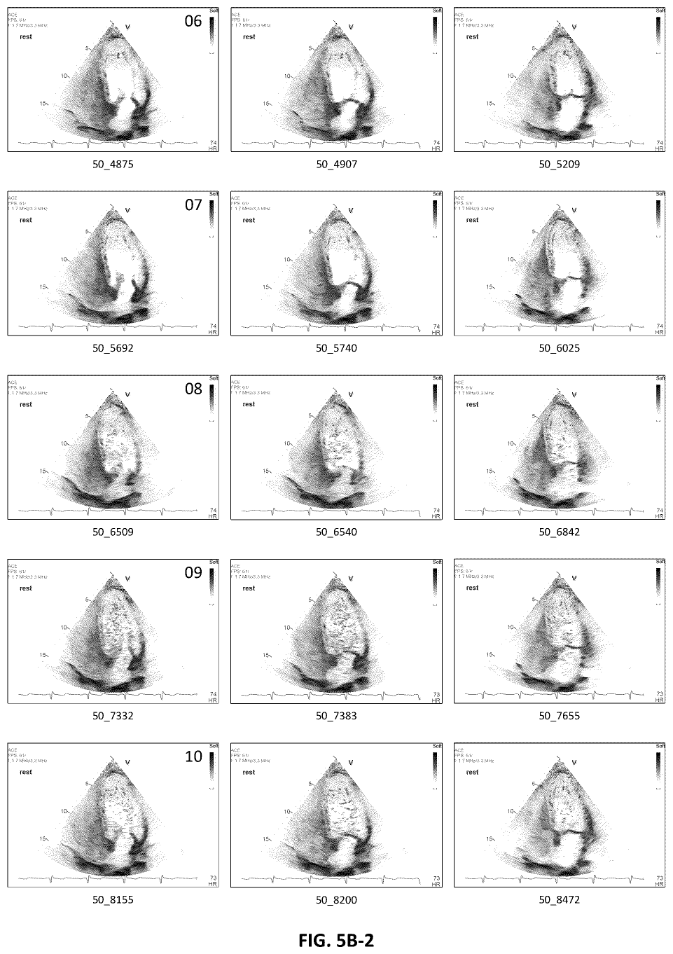

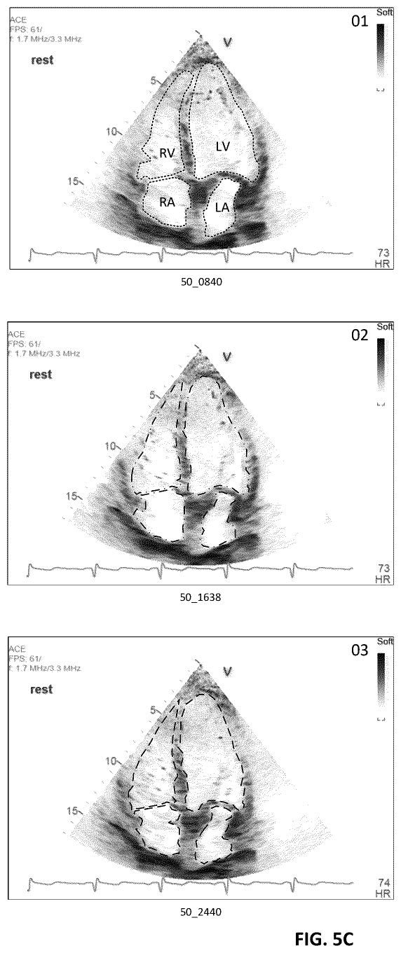

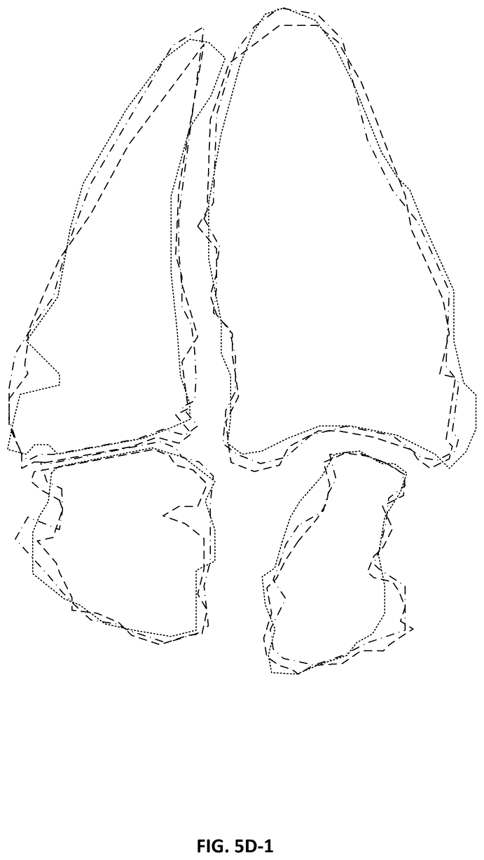

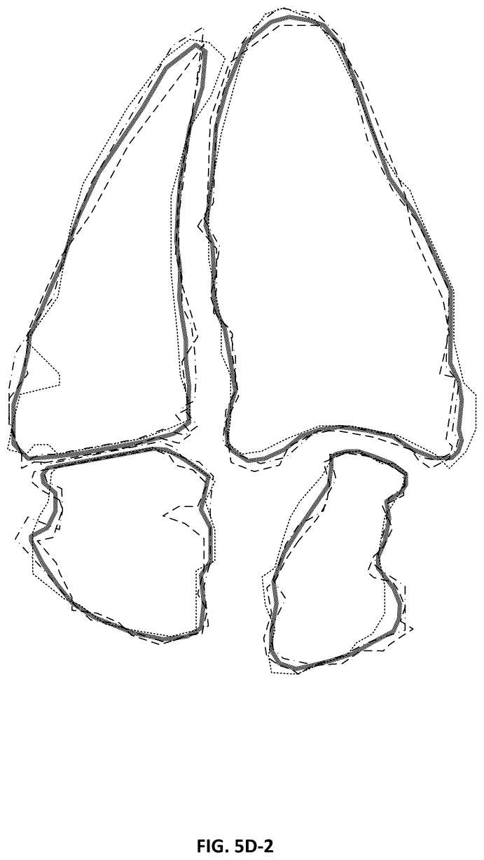

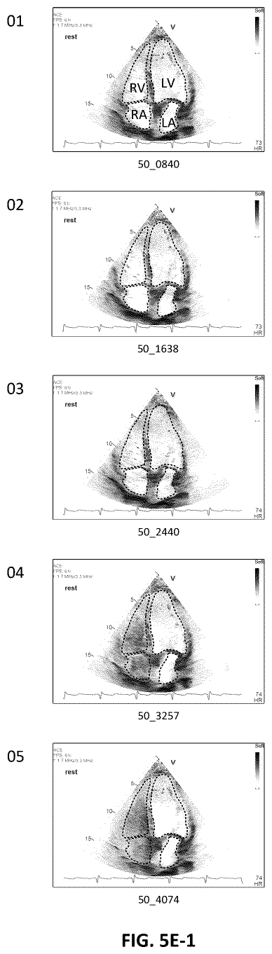

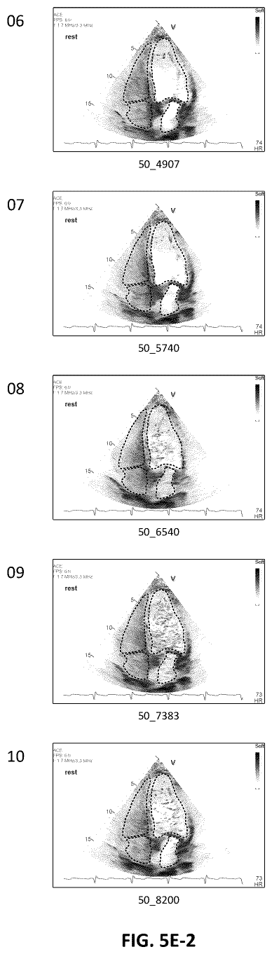

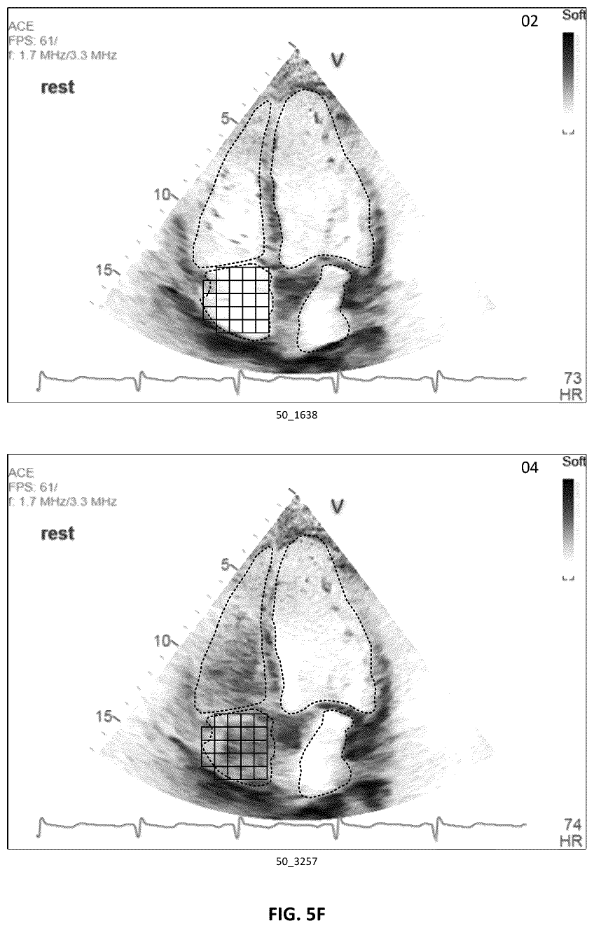

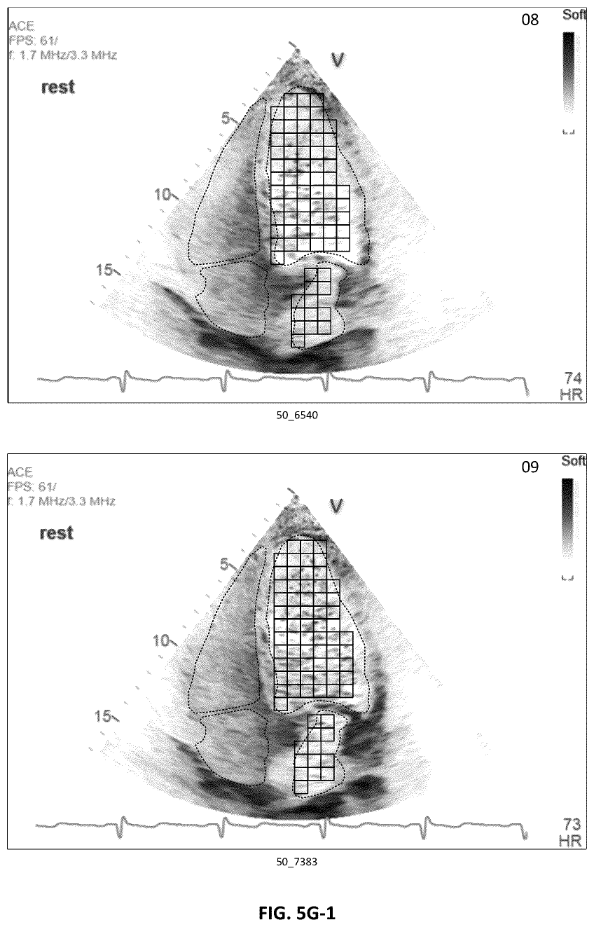

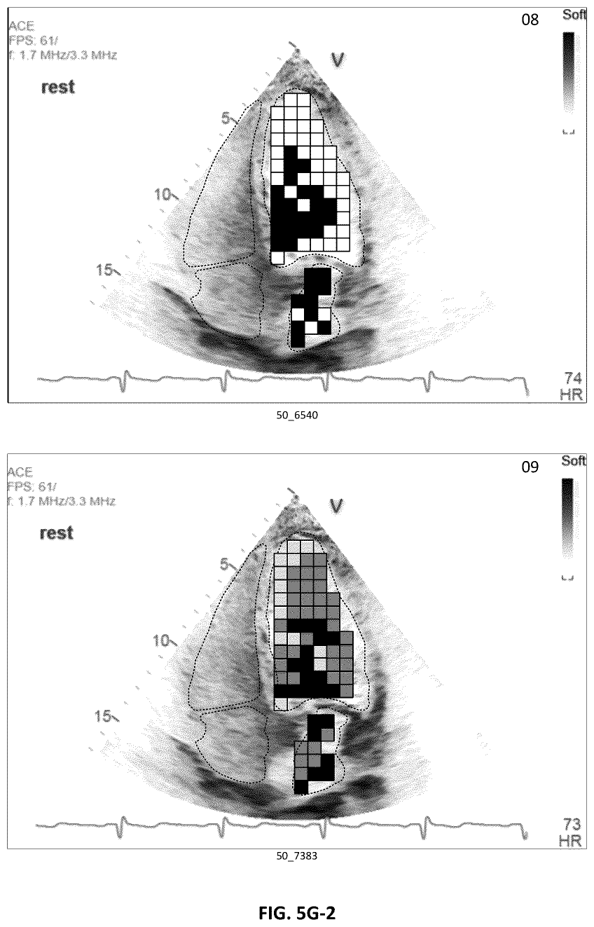

FIG. 5A illustrates an ECG waveform that corresponds to a sequence of ultrasound images from a patient (“patient 50” in one study of fifty echocardiograms) that will be illustrated and described with reference to figures that follow. As illustrated, the ECG waveform comprises 13 cardiac cycles (labeled). Timestamps that will be referenced in following figures are provided—in particular, time stamps associated with the start of the P wave, the start of the Q wave, and the start of the T wave.

FIGS. 5B-1 and 5B-2 illustrate ultrasound images corresponding to ten of the cardiac cycles captured in the ECG waveform of FIG. 5A. For clarity, as illustrated, black and white have been inverted relative to a standard ultrasound image—that is, ultrasound images are illustrated herein as black on white, rather than the standard white on black ultrasound image. Individual image labels in FIGS. 5B-1 and 5B-2 correspond to the time stamps shown in FIG. 5A. The ultrasound images were taken during a bubble study of “patient 50” using a TTE procedure. As indicated in FIG. 5A, microbubbles were injected into patient 50, and these bubbles were first seen on the right side of the patient's heart during cycle 04; bubbles were seen on the left side of the patient's heart during cycle 07—indicating a shunt in the patient's heart.

In the images shown in FIGS. 5B-1 and 5B-2, bubbles are visible to a human eye on the right side of the patient's heart. In some implementations, the presence of such bubbles can be detected automatically, for example, through an exemplary method that is now described.

FIG. 5C illustrates three snapshots in time, corresponding to substantially the same point in three different cardiac cycles (e.g., as shown, approximately at the end of the QRS phase). (As used herein, “substantially” or “about” or “approximately” may refer to within 1% or 5% or 10% or 25% of a nominal value; with respect to cardiac cycles, the same terms may refer to very near to an identified portion of a phase, when considered relative to the time period(s) of that phase and adjacent phases—for example, “approximately at the end of the QRS phase” may refer to the last 1-25% of the QRS phase or the first 1-5% of the ST phase.)

Multiple images corresponding to the same point of different cardiac cycles may be analyzed to delineate different structures of the heart. FIG. 5C illustrates outlines corresponding to the four chambers of a human heart—the right ventricle (“RV”), left ventricle (“LV”), left atrium (“LA”) and right atrium (“RA”). These chambers may be identified through image analysis of each ultrasound snapshot. That is, in some implementations, a grayscale or color value for each pixel or cluster of pixels may be analyzed relative to adjacent pixels; and any pixel or cluster of pixels that is lighter than (or darker than) at least one adjacent pixel or cluster of pixels (e.g., by some threshold value) may be identified as corresponding to a cardiac chamber, rather than cardiac tissue. Boundaries may then be identified for each chamber, according to analysis of a given ultrasound image—as depicted in FIG. 5C.

Boundaries from different cardiac cycles may be averaged together—as depicted in FIGS. 5D-1 and 5D-2. Average boundaries may then be superimposed on the original images to facilitate further analysis—as depicted in FIGS. 5E-1 and 5E2.

Described herein is pixel-by-pixel analysis that compares grayscale or color values between pixels and relative to a threshold value. The threshold value may vary based on location within the heart. For example, some ultrasound images may capture the atria more clearly than the ventricles; thus, threshold values used in delineating chambers from tissue may be different for the atria than for the ventricles. In some implementations, the threshold may vary across a given chamber. For example, for images such as those shown in FIGS. 5B-1 and 5B-2—where the atria and the portion of the ventricles that are closest to the atria are clearer than the distal portions of the ventricles—thresholds may vary from one end of the heart to the other. Moreover, thresholds may be determined for each patient, based, for example, on specific color or grayscale values at particular points (anatomical and time-based) within a given patient's echocardiogram images. For example, a patient's interatrial septum may be used to establish one reference threshold (e.g., black, in the inverted images presented herein), and the middle of the left atrium, prior to injection of bubbles, could be used to establish another reference threshold (e.g., white)—against which thresholds other regions at different points of time could be compared).

In some implementations, machine learning may be applied to distinguish chambers from tissue. In some implementations, only portions of chamber(s) may be delineated. For example, it may only be necessary to delineate atria and portions of one or both of the ventricles that are closest to the atria. In some implementations, delineated structures may be associated with easily identifiable landmarks (e.g., for superposition onto other images at different points in time, to facilitate analyses as described herein)—for example, to points on the exterior heart wall, to portions of the atrial septum, to specific valves that are visible and identifiable at different points in the cardiac cycle, etc.

Regardless of the method employed to identify “regions of interest” (e.g., certain heart chambers), further analysis may be performed within the identified regions of interest. To further depict two analyses, FIGS. 5E-1 and 5E-2 illustrate the ultrasound images of FIGS. 5B-1 and 5B-2, with the regions of interest (as shown, each of the four heart chambers) highlighted.

One analysis that may be advantageous is confirmation of sufficient opacification of the right atrium during a bubble study. Turning to FIG. 5F, ultrasound images of the patient's heart are shown for cycle 02 (a reference, pre-bubble cycle) and cycle 04 (the cycle in which bubbles are first detected in the right atrium). A method may be employed whereby specific subsets of the right atrium are analyzed (e.g., square segments, as illustrated), and colors or grayscale pixel values are compared between different cardiac cycles. The right atrium is shown opacified at cycle 04, and this level of opacification may be quantitatively compared to a threshold value to, in some implementations, confirm sufficient opacification. Moreover, in some implementations, the right atrium is analyzed over multiple cardiac cycles to confirm that it remains opacified for a sufficient number of cycles (e.g., five cycles, in some implementations).

In some implementations, sufficient opacification may be specifically confirmed adjacent to the right atrial septum (e.g., to rule out situations such as those involving patients with Eustachian valves that direct blood from the inferior vena cava (IVC) to the interatrial septum, thereby “washing out” bubbles that are coming from the superior vena cava (SVC)). In some implementations, it may be advantageous to detect “negative contrast” in an otherwise opacified right atrium—which may indicate a significant left-to-right shunt that is sometimes seen with ASDs.

If sufficient opacification (e.g., sufficient level of opacification, for a sufficient number of cardiac cycles) is confirmed, additional analyses may be performed. For example, the left atrium may be analyzed to detect presence of any bubbles within three to five cardiac cycles of when the bubbles first appear on the right side of the heart. FIG. 5G-1 illustrates two cardiac cycles (cycle 08 and cycle 09) during which bubbles are detected in both the left atrium and the left ventricle.

In some implementations, bubbles may be assessed by analysis of grayscale color of individual pixels or clusters or pixels within a region of interest. In some implementations, as depicted in FIG. 5G-2, values associated with clusters of pixels (e.g., the small square segments illustrated) may be averaged and converted to one of a finite number of values (e.g., to facilitate more efficient processing, or to graphically depict or highlight quantities of detected bubbles). For example, clusters of pixels may be converted to one of two binary values (e.g., black or white, as shown in cycle 08 in FIG. 5G-2) or one of multiple different grayscale values (e.g., black, gray, light gray, as shown in cycle 09 in FIG. 5G-2).

In some implementations, color or grayscale values associated with certain numbers of segments within a region of interest or with specific anatomical segments may be employed to “score” a detected number of bubbles, and by extension, a corresponding shunt. For example, in some implementations, a greater number of segments within a region of interest that are determined to include bubbles may be scored higher than a smaller number of bubble-containing segments within the same region of interest. Such a score may then be used to indicate severity of a corresponding shunt. In some implementations, shunts that are associated with high scores may be reviewed for possible surgical repair; shunts that are associated with low scores may be monitored and/or treated with medicine (e.g., blood thinners); and shunts that are associated with intermediate scores may be subjected to additional testing (e.g., a follow-on TEE study, if the present study was a TTE study).

In some implementations, the anatomical location of detected bubbles may influence scoring. For example, detected bubbles very near an atrial wall may be scored lower than bubbles detected at an end of the atrium opposite the atrial septum. As another example, bubbles that are detected in the left ventricle (as shown in FIGS. 5G-1 and 5G-2) may be scored higher than bubbles that are contained within the left atrium. As another example, bubbles that are detected entering the left atrium near the pulmonary veins may be scored in a manner that suggests a transpulmonary shunt. Bubbles that are detected on the left side may be further analyzed relative to time—for example, to differentiate a low-level steady trickle versus a bolus of bubbles appearing spontaneously after a cough or performance of the Valsalva maneuver (with a detected bolus being scored higher, in some implementations, than a low-level trickle). Various scoring methods are contemplated and possible.

Another analysis that may be automated, in some implementations, is confirmation that a Valsalva maneuver was performed property. A Valsalva maneuver (where the patient forces expiration against a closed glottis, to increase intrathoracic pressure and increase venous return upon release of the maneuver) is often performed in conjunction with a bubble study in order to increase right atrial pressure—so as to overcome some of the natural resistance, when a shunt is present, to the passage of blood from right atrium to left atrium (given the inherently higher pressure in the left atrium, without performance of a Valsalva maneuver). Performance of such a maneuver can illuminate shunts that might not otherwise be detectible; such illumination, however, can be dependent on the quality of the maneuver—which ideally (depending on how flexible the patient's septum is) should result in a visible right-to-left shift in the interatrial septum (or visible decrease in left atrium volume). Thus, it can be advantageous to confirm proper performance of the maneuver. In some implementations, coughing during specific periods of a bubble study may have a similar effect as performing a Valsalva maneuver; and certain techniques described herein for confirming proper execution of a Valsalva maneuver may also be employed to confirm desired effect of coughing. Other methods of increasing right-side pressure may also be employed and analyzed.

In some implementations, a method to confirm proper performance of a Valsalva maneuver can analyze right atrium volume changes during performance of the maneuver, relative to a baseline; and confirm either a changed volume of the right atrium or a detectible shift in the interatrial septum. One such exemplary method is now illustrated and described with reference to FIGS. 6A-1, 6A-2, 6A-3; 6B-1, 6B-2, 6B-3; and 6C.

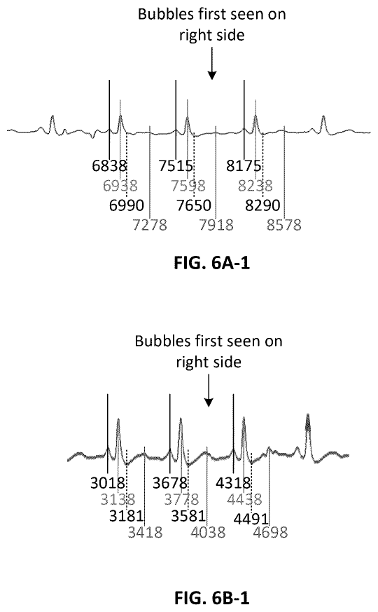

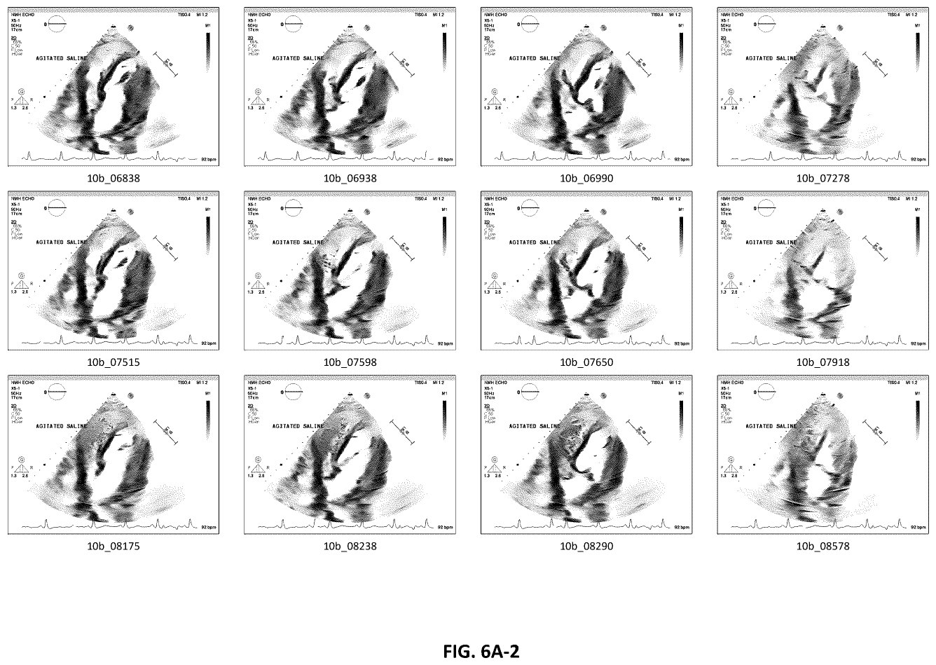

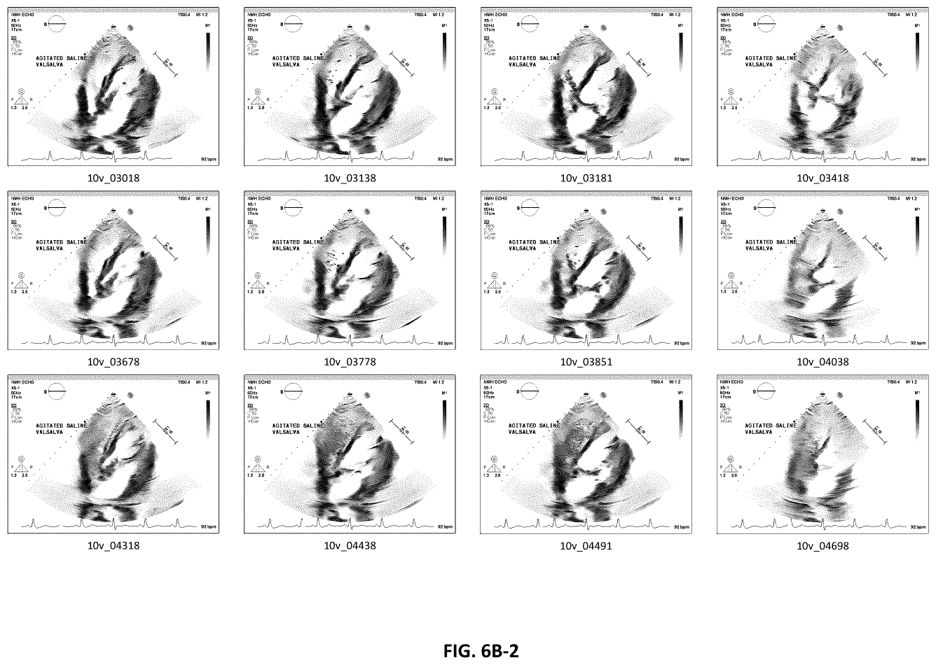

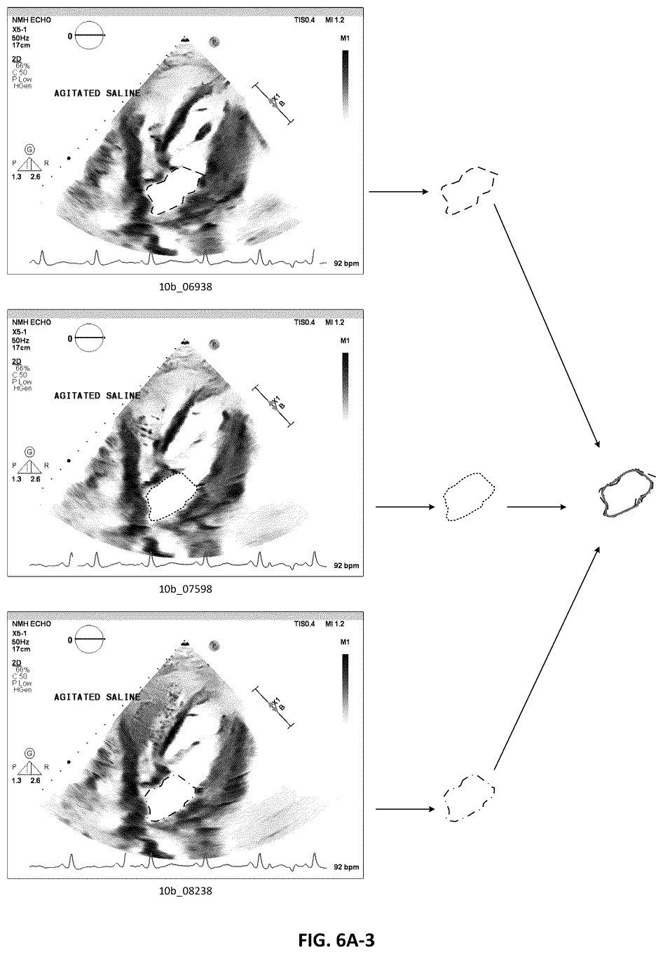

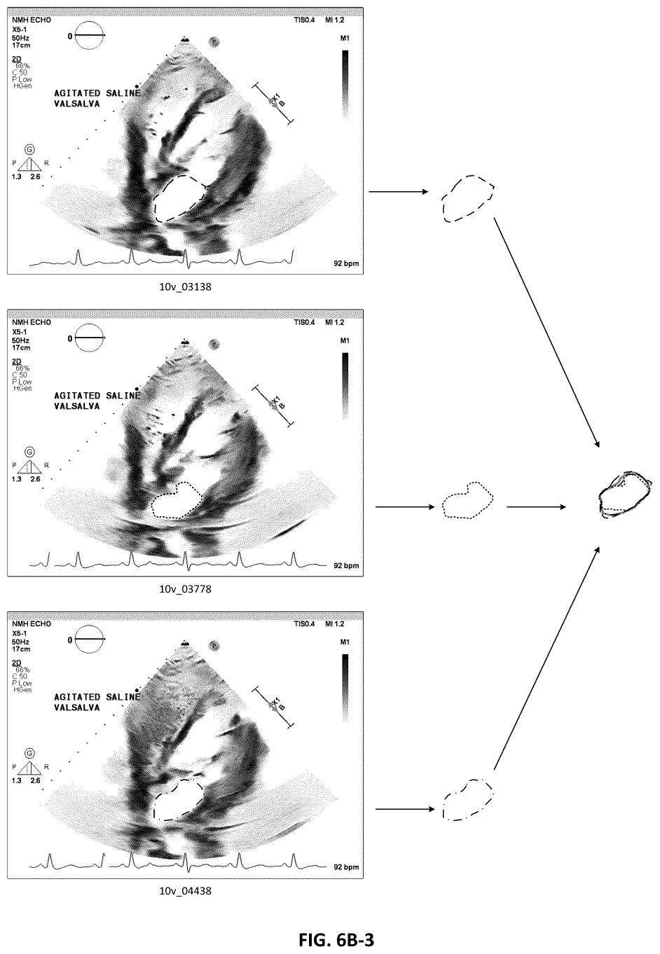

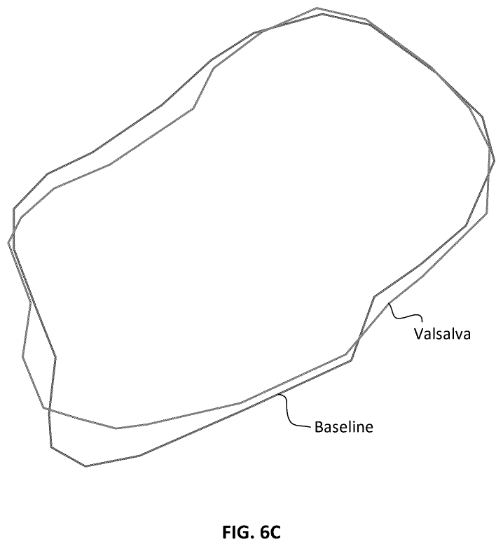

FIG. 6A-1 illustrates a baseline cardiac waveform snapshot for a patient who underwent a bubble study. In the waveform shown, bubbles were injected and detected; but the patient did not perform a Valsalva maneuver. FIG. 6A-2 illustrates corresponding echocardiogram images. Time points are correlated between the images of FIG. 6A-2 and the waveform of FIG. 6A-1. FIG. 6A-3 depicts an analysis of three specific images from the same phase of the patient's cardiac cycle. In particular, FIG. 6A-3 depicts identification of the boundary of the left atrium in each of the images. Further depicted is extraction of the identified boundary from each image and averaging of that boundary to determine an average baseline boundary.

A similar process is depicted for detecting a boundary of the left atrium during a Valsalva maneuver—FIG. 6B-1 illustrates a cardiac waveform snapshot for a patient who underwent a bubble study (while performing a Valsalva maneuver); FIG. 6B-2 illustrates corresponding echocardiogram images (again, correlated to the waveform by the illustrated time points). FIG. 6B-3 depicts an analysis of three specific images from the same phase of the patient's cardiac cycle to determine an average boundary for the left atrium during the Valsalva maneuver. FIG. 6C illustrates a comparison of the two boundaries—which comparison may be further analyzed to determine whether an expected shift in the interatrial septum or another portion of an atrial wall is seen or an expected decrease in left atrium volume. If not, real time feedback may be provided, such that the procedure may be repeated.

Described and illustrated are methods to identify boundaries of the left atrium. But similar techniques could be applied to analyze portions of the atrial septum, or other parameters that can analyzed over time to confirm proper execution of the Valsalva maneuver. For example, ECG data may be analyzed to help confirm proper execution of the Valsalva maneuver (for example, by analyzing the time of the P wave, the amplitude of the P wave, the RR interval, the PR time, the QTc interval, T wave amplitude, T/R amplitude, etc.—parameters that may change during or immediately after a Valsalva maneuver).

Automated image processing, as described herein, may be performed on one or a plurality of machines or devices. For example, portions of methods described here may be performed within onboard computing resources of an ultrasound machine itself; and certain information about the images (e.g., quality and duration of opacification or detection of bubbles; presence of detected bubbles on the left side of the heart; a score associated with such bubble detection; confirmation that a Valsalva or other maneuver resulted in a detectible shift of the interatrial septum; etc.) may be available in real time or nearly in real time. As another example, images may be captured by a bedside ultrasound machine that is proximate a patient undergoing a test, and video and image media may be exported to a supplemental computing device that is also proximate the patient (e.g., a laptop of tablet computing device used by an ultrasound clinician). Certain aspects of the image processing described herein may be performed on that computing device and available with only a short delay (e.g., delay associated with some processing time and the time necessary to transfer media to the supplemental computing device). As another example, media may be captured by an ultrasound machine and transferred via a network to a remote computing device for analysis—either in real time, or for later post-processing. Implementations in which real time processing is possible may be advantageous; as it may be able to identify deficiencies in a procedure aspect while the patient is still present and while the procedure can be easily repeated—for example, a failed or insufficient Valsalva maneuver, or poor or limited-duration opacification on the left side.

While several implementations have been described with reference to exemplary aspects, it will be understood by those skilled in the art that various changes may be made and equivalents may be substituted for elements thereof without departing from the contemplated scope. For example, described herein are methods for characterizing cardiac shunts. The methods could easily be adapted, however, to identify shunts or defects outside of the heart. For example, multiple bubble studies with progressively larger bubbles may be employed to detect and assess pulmonary shunts, aneurysms, plaque buildup or other narrowing of vessels, spasm or constriction of vessel, or other conditions. Techniques described herein could be employed with contrast agents other than bubbles—for example, with Perflutren Protein-Type A Microspheres or other contrast agents that may enhance contrast and improve delineation of left ventricular endocardial borders. References are made to TTE; but techniques described here could be applied in the context of TEE procedures as well. Automated image analysis could be applied to calculate hemodynamics in addition to detecting shunts. In addition, many modifications may be made to adapt a particular situation or material to the teachings provided herein without departing from the essential scope thereof. Therefore, it is intended that the scope not be limited to the particular aspects disclosed but include all aspects falling within the scope of the appended claims.

What is claimed is:

1. A method comprising:
performing an echocardiogram procedure on a patient to generate a plurality of echocardiogram images associated with different points within a cardiac cycle of the patient, across multiple cardiac cycles of the patient;
during the echocardiogram procedure, intravenously directing bubbles into a patient's venous circulatory system and to a right side of the patient's heart;
prior to detecting presence of the bubbles on the right side of the patient's heart, and from a subset of echocardiogram images within the plurality of echocardiogram images, each echocardiogram image in the subset corresponding substantially to a same point in different cardiac cycles, identifying structures of the patient's heart, including at least a left atrium, a right atrium and an atrial septum, and identifying composite boundaries for the structures;
after detecting presence of the bubbles on the right side of the patient's heart, comparing opacification of the right atrium to a threshold level of opacification to confirm sufficient opacification of the patient's right atrium;
analyzing additional echocardiogram images in the plurality of echocardiogram images relative to the composite boundaries to detect presence of the bubbles on a left side of the patient's heart;
based on analyzing the additional echocardiogram images, calculating a score based on a nature of detected bubbles on the left side of the patient's heart, wherein a higher value for the score corresponds to a relatively more severe shunt and a lower value for the score corresponds to a relatively less severe shunt;
based on the score initiating treatment in the patient; wherein initiating treatment comprises, (i) for a high score, surgically closing the shunt; (ii) for a low score, treating the patient with blood thinners; and (iii) for an intermediate score, performing a follow-on transesophageal echocardiogram (TEE) study.

2. The method of claim 1, wherein intravenously directing the bubbles comprises injecting an agitated solution of saline or dextrose into a median cubital vein of the patient.

3. The method of claim 1, wherein performing an echocardiogram procedure comprises performing either a transthoracic echocardiogram (TTE) or a TEE.

4. The method of claim 1, wherein surgically closing the shunt in the patient's heart comprises using a catheter-delivered percutaneous closure device.

5. The method of claim 1, further comprising determining a blood flow rate, using ultrasound imaging, by monitoring movement of the bubbles within the patient's heart.

6. The method of claim 1, wherein analyzing the additional echocardiogram images relative to the composite boundaries comprises segmenting a region of interest and detecting the bubbles in one or more segments within the region of interest.

7. The method of claim 6, wherein the region of interest comprises a portion of the left atrium and a component of the calculated score is associated with proximity of detected bubbles relative to the atrial septum, wherein the component is higher for detected bubbles farther from the atrial septum than for detected bubbles that are closer to the atrial septum.

8. The method of claim 6, wherein the region of interest comprises a portion of the left atrium and a component of the calculated score is based on a time analysis of detected bubbles, wherein the component is higher for a bolus of detected bubbles within a smaller window of time relative to a smaller number of detected bubbles over a longer window of time.

9. The method of claim 6, wherein identifying the structures of the heart further comprises identifying a left ventricle, wherein the region of interest comprises the left ventricle, wherein a component of the score is based on whether bubbles are detected in the left ventricle, and wherein the component is higher when the bubbles are detected in the left ventricle and lower when the bubbles are not detected in the left ventricle.

10. The method of claim 1, wherein confirming sufficient opacification of the patient's right atrium further comprises confirming sufficient opacification adjacent the atrial septum.

11. The method of claim 1, wherein confirming sufficient opacification of the patient's right atrium further comprises confirming absence of negative contrast adjacent the atrial septum.

12. The method of claim 1, wherein the additional echocardiogram images comprise echocardiogram images that are captured within three to five cardiac cycles of when the bubbles are detected on the right side of the patient's heart.

13. The method of claim 1, further comprising confirming proper execution of a Valsalva maneuver.

14. The method of claim 13, wherein confirming proper execution of a Valsalva maneuver comprises confirming, using at least two images in the echocardiogram images, that the atrial septum has shifted from the right atrium toward the left atrium.

15. The method of claim 13, wherein confirming proper execution of a Valsalva maneuver comprises confirming, using at least two images in the echocardiogram images, that the right atrium has increased in volume or that the left atrium has decreased in volume.

16. A method comprising:
performing an echocardiogram procedure on a patient to generate a plurality of echocardiogram images associated with different points within a cardiac cycle of the patient, across multiple cardiac cycles of the patient;
during the echocardiogram procedure, intravenously directing bubbles into a patient's venous circulatory system and to a right side of the patient's heart;
prior to detecting presence of the bubbles on the right side of the patient's heart, and from a subset of echocardiogram images within the plurality of echocardiogram images, each echocardiogram image in the subset corresponding substantially to a same point in different cardiac cycles, identifying structures of the patient's heart, including at least a left atrium, a right atrium and an atrial septum, and identifying composite boundaries for the structures;
after detecting presence of the bubbles on a right side of the patient's heart, comparing opacification of the right atrium to a threshold level of opacification to confirm sufficient opacification of the patient's right atrium;
analyzing supplemental echocardiogram images in the plurality of echocardiogram images relative to the composite boundaries to confirm at least one of a shift in the atrial septum from the right atrium toward the left atrium, an increase in volume of the left atrium or a decrease in volume of the right atrium;
analyzing additional echocardiogram images in the plurality of echocardiogram images relative to the composite boundaries to detect presence of the bubbles on a left side of the patient's heart;
based on analyzing the additional echocardiogram images, calculating a score based on a nature of detected bubbles on the left side of the patient's heart, wherein a higher value for the score corresponds to a relatively more severe shunt and a lower value for the score corresponds to a relatively less severe shunt;
based on the score, initiating treatment in the patient; wherein initiating treatment comprises, for a high score, surgically closing the shunt; for a low score, treating the patient with blood thinners; and for an intermediate score, performing a follow-on transesophageal echocardiogram (TEE) study.
